(19) United States
(12) Patent Application Publication
(10) Pub. No.: US 2026/0327682 A1
(43) Pub. Date: Oct. 22, 2026

(54) CHEMICAL LIGATION TECHNIQUES

(71) Applicant: President and Fellows of Harvard College, Cambridge, MA (US)

(72) Inventors: Xiaowei Zhuang, Cambridge, MA (US); Aditya Venkatramani, Cambridge, MA (US)

(73) Assignee: President and Fellows of Harvard College, Cambridge, MA (US)

(21) Appl. No.: 19/474,029

(22) PCT Filed: Apr. 10, 2024
(86) PCT No.: PCT/US2024/023812

§ 371 Date: Oct. 9, 2025

Related U.S. Application Data
(60) Provisional application No. 63/458,610, filed on Apr. 11, 2023.
(60) Provisional application No. 63/506,294, filed on Jun. 5, 2023.
(60) Provisional application No. 63/506,337, filed on Jun. 5, 2023.

(51) Int. Cl. C12Q 1/6806

(52) U.S. Cl. CPC C12Q 1/6806

(57) ABSTRACT

The present disclosure generally relates to systems and methods for chemical ligation. Certain aspects as discussed herein are generally directed to applying light to cleave nucleic acids containing photocleavable linkers and/or protection groups, either of which may include a photocleavable blocking group. In some embodiments, the photocleaving exposes a 5′-phosphate, which can be used for subsequent reactions, such as ligase reactions. In some embodiments, the photocleaving exposes an OH group on the 5′-end, which can be phosphorylated in some embodiments, resulting in an exposed 5′-phosphate. In some embodiments, additional nucleic acids, which may contain photocleavable linkers or protection groups, may be attached to the exposed 5′-phosphate. In some cases, this process may be repeated one or more times, e.g., resulting in a plurality or barcode of nucleic acids. In certain cases, the nucleic acids may be attached to a sample or a surface, and such reactions may be controlled, e.g., to form or synthesize barcodes in specific spatial locations in the sample.

### RELATED APPLICATIONS

This application claims the benefit of U.S. Provisional Patent Application Ser. No. 63/506,337, filed Jun. 5, 2023, entitled “Chemical Ligation Techniques,” by Zhuang, et al.; U.S. Provisional Patent Application Ser. No. 63/458,610, filed Apr. 11, 2023, entitled “Multiplexed Optical Barcoding for Spatial Omics,” by Zhuang, et al.; and U.S. Provisional Patent Application Ser. No. 63/506,294, filed Jun. 5, 2023, entitled “Multiplexed Optical Barcoding for Spatial Omics,” by Zhuang, et al., each of which is incorporated herein by reference in its entirety.

### GOVERNMENT FUNDING

This invention was made with government support under NS116593 awarded by National Institutes of Health (NIH). The government has certain rights in this invention.

### FIELD

The present disclosure generally relates to systems and methods for chemical ligation.

### BACKGROUND

With the development of high-throughput sequencing technology, it is now possible to sequence huge numbers of nucleic acids removed or extracted from a sample. For example, a single “run” of an Illumina HiSeq sequencer (HiSeq 2500 Rapid Run Mode) takes 27 hours, and generates-1.2 billion paired end reads (reactions). However, generating the huge numbers of nucleic acids to encode information and that can be readily distinguished using such high-throughput sequencing technology is still difficult. Many researchers tag or “barcode” samples prior to sequencing, so that many samples can be analyzed in a single sequencing run. Such barcoding techniques, however, have proven difficult to scale up, and accordingly, improvements in systems and methods for chemical ligation are still needed.

### SUMMARY

The present disclosure generally relates to systems and methods for chemical ligation. The subject matter of the present disclosure involves, in some cases, interrelated products, alternative solutions to a particular problem, and/or a plurality of different uses of one or more systems and/or articles.

Certain aspects are generally drawn to methods. For example, in one set of embodiments, the method comprises providing a first nucleic acid comprising a photocleavable linker and a spacer sequence, applying light to cleave the photocleavable linker to expose a 5′-phosphate and remove the spacer sequence from the nucleic acid, and attaching a second nucleic acid to the 5′-phosphate.

The method, in accordance with another set of embodiments, comprises providing a first nucleic acid comprising a photocleavable protecting group, applying light to cleave the photocleavable protecting group to expose a 5′-OH, phosphorylating the 5′-OH to a 5′ phosphate, and attaching a second nucleic acid to the 5′-phosphate.

In yet another set of embodiments, the method comprises providing a first nucleic acid comprising a first photocleavable blocking group; applying light to the sample to cleave the first photocleavable blocking group; attaching a second nucleic acid to the first nucleic acid, wherein the second addition sequence comprises a second photocleavable blocking group; applying light to the sample to cleave the second photocleavable blocking group; and attaching a third nucleic acid to the second nucleic acid.

In accordance with still another set of embodiments, the method comprises providing a first nucleic acid comprising a first photocleavable blocking group; applying light to the sample to cleave the first photocleavable blocking group; exposing the first nucleic acid to a DNA splint comprising a first portion and a second portion, wherein the first portion binds to at least a portion of the first nucleic acid; exposing the splint sequence to a second nucleic acid, wherein the second portion binds to at least a portion of the second nucleic acid; and attaching the first nucleic acid to the second nucleic acid.

The method, in yet another set of embodiments, comprises providing a first nucleic acid comprising a photocleavable linker and a spacer sequence, applying light to cleave the photocleavable linker and remove the spacer sequence from the nucleic acid, and attaching a second nucleic acid to the first nucleic acid using a DNA ligase. In some embodiments, the spacer sequence, when present, inhibits the DNA ligase from attaching nucleic acids.

In another set of embodiments, the method comprises attaching a nucleic acid in a first location but not a second location within a sample. In certain cases, the nucleic acid may comprise a photocleavable blocking group.

According to still another set of embodiments, the method comprises attaching nucleic acids to different locations within a sample using a DNA ligase. In some embodiments, some or all of the nucleic acids may comprise a blocking group that inhibits the DNA ligase from binding additional nucleotides to the DNA.

In another aspect, the present disclosure encompasses methods of making one or more of the embodiments described herein. In still another aspect, the present disclosure encompasses methods of using one or more of the embodiments described herein.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 is a schematic illustrating tagging of mRNA and/or proteins, spatial barcoding, extraction, and sequencing, in one embodiment;

FIG. 2 is a schematic illustrating introducing DNA tags into a sample, in another embodiment;

FIG. 3 illustrates an example 2-dimensional region of space has been discretized, in yet another embodiment;

FIG. 4 is a schematic illustrates barcodes uniquely mapping different spatial positions within a sample, in still another embodiment;

FIG. 5 illustrates a recursive algorithm for generating barcodes, in yet another embodiment;

FIG. 6 is an assay showing ligation and photocleaving efficiency, in one embodiment;

FIG. 7 is an assay showing ligation of a DNA tag, in another embodiment;

FIG. 8 is a histogram showing melting temperatures of splints, in still another embodiment;

FIG. 9 is shows a ligation assay, in yet another embodiment; and

FIG. 10 shows an assay used for determining efficiency, in still another embodiment.

FIG. 11 illustrates the tape station results for 1, 2, 3, and 4 ligations, with >95% efficiency per ligation, in one embodiment;

FIG. 12 is an image showing the fluorescent signal in the photocleaved region, in another embodiment;

FIG. 13 is a DAPI signal showing the presence of cells, in yet another embodiment;

FIGS. 14A-14B show the mRNA content of tissue samples captured using in-situ Reverse Transcription (RT), in still another embodiment;

FIG. 15 is a schematic illustrating a ligation order, in one embodiment;

FIG. 16 shows a DAPI image, in another embodiment; and

FIGS. 17A-17B illustrate results of sequencing mapping onto mouse and human genomes, in yet another embodiment.

### BRIEF DESCRIPTION OF THE SEQUENCES

Below is a list of DNA sequences used for certain embodiments and examples herein.

| | | SEQ ID |
|---|---|---|
| Type | Sequence | NO: |
| DNA tag | /5Phos/ TGTGT TAGGT ATGGA AGAGA TTTTT /36-FAM/ | 1 |
| DNA tag | /5Phos/ TGTGT TAGGT ATGGA AGAGA TTTTT TTTTT TTTTT | 2 |
| | TTTTT TTTTT TTTTT TTTTT TTTTT AATCAGCCATACCACATTTG | |
| DNA tag | /5Phos/ AGAGA ATGGA TAGGT TGTGT TTTTT TTTTT TTTTT | 3 |
| | TTTTT TTTTT TTTTT TTTTT TTTTT AATCAGCCATACCACATTTG | |
| DNA tag | /5Phos/ TGTGT TAGGT ATGGA AGAGA TT +TT +TT +TT +TT | 4 |
| | +TT +TT +TT +TT +TT +TT +TTT AATCAGCCATACCACATTTG | |
| DNA letter | TTTTTTTTTTTTTTTTTTTT/iSpPC/AGAGA | 5 |
| DNA letter | TTTTTTTTTTTTTTTTTTTT/iSpPC/ATGGA | 5 |
| DNA letter | TTTTTTTTTTTTTTTTTTTT/iSpPC/TAGGT | 5 |
| DNA letter | TTTTTTTTTTTTTTTTTTTT/iSpPC/TGTGT | 5 |
| DNA letter | GTAAAACGACGGCCAG/iSpPC/AGAGA | 6 |
| DNA letter | GTAAAACGACGGCCAG/iSpPC/ATGGA | 6 |
| DNA letter | GTAAAACGACGGCCAG/iSpPC/TAGGT | 6 |
| DNA letter | GTAAAACGACGGCCAG/iSpPC/TGTGT | 6 |
| PCR | GGATTATTCATACCGTCCCA | 7 |
| primer | | |
| PCR | CAAATGTGGTATGGCTGATT | 8 |
| primer | | |
| Splints for | ACCTATCCATTCTCT | 9 |
| letter | ACACATCCATTCTCT | 10 |
| | TCCATACCTATCTCT | 11 |
| | ACACAACCTATCTCT | 12 |
| | TCCATACACATCTCT | 13 |
| | ACCTAACACATCTCT | 14 |
| | ACCTATCTCTTCCAT | 15 |
| | ACACATCTCTTCCAT | 16 |
| | TCTCTACCTATCCAT | 17 |
| | ACACAACCTATCCAT | 18 |
| | TCTCTACACATCCAT | 19 |
| | ACCTAACACATCCAT | 20 |
| | TCCATTCTCTACCTA | 21 |
| | ACACATCTCTACCTA | 22 |
| | TCTCTTCCATACCTA | 23 |
| | ACACATCCATACCTA | 24 |
| | TCTCTACACAACCTA | 25 |
| | TCCATACACAACCTA | 26 |
| | TCCATTCTCTACACA | 27 |
| | ACCTATCTCTACACA | 28 |
| | TCTCTTCCATACACA | 29 |
| | ACCTATCCATACACA | 30 |
| | TCTCTACCTAACACA | 31 |
| | TCCATACCTAACACA | 32 |
| Splint for | TCC ATT CTC TTG GGA CGG TA | 33 |
| primer | ACC TAT CTC TTG GGA CGG TA | 34 |
| | ACA CAT CTC TTG GGA CGG TA | 35 |
| | TCT CTT CCA TTG GGA CGG TA | 36 |
| | ACC TAT CCA TTG GGA CGG TA | 37 |
| | ACA CAT CCA TTG GGA CGG TA | 38 |
| | TCT CTA CCT ATG GGA CGG TA | 39 |
| | TCC ATA CCT ATG GGA CGG TA | 40 |
| | ACA CAA CCT ATG GGA CGG TA | 41 |
| | TCT CTA CAC ATG GGA CGG TA | 42 |
| | TCC ATA CAC ATG GGA CGG TA | 43 |
| | ACC TAA CAC ATG GGA CGG TA | 44 |

### DETAILED DESCRIPTION

The present disclosure generally relates to systems and methods for chemical ligation. Certain aspects as discussed herein are generally directed to applying light to cleave nucleic acids containing photocleavable linkers or protection groups, either of which may include a photocleavable blocking group. In some embodiments, the photocleaving exposes a 5′-phosphate, which can be used for subsequent reactions such as ligase reactions. In some embodiments, the photocleaving exposes a OH group on the 5′-end, which can be phosphorylated in some embodiments, resulting in an exposed 5′-phosphate. In some embodiments, additional nucleic acids, which may contain photocleavable linkers or protection groups, etc., may be attached to the exposed 5′-phosphate. In some cases, this process can be repeated one or more times, e.g., resulting in a plurality or barcode of nucleic acids. In certain cases, the nucleic acids may be attached to a sample or a surface, and such reactions may be controlled, e.g., to form or synthesize barcodes in specific spatial locations in the sample.

One aspect is generally directed to systems and methods for producing nucleic acids using photocleavable linkers or protection groups that can be cleaved using light (e.g., ultraviolet light) and which can, in certain embodiments, be used for subsequent ligase reactions. Non-limiting examples of such photocleavable linkers include:

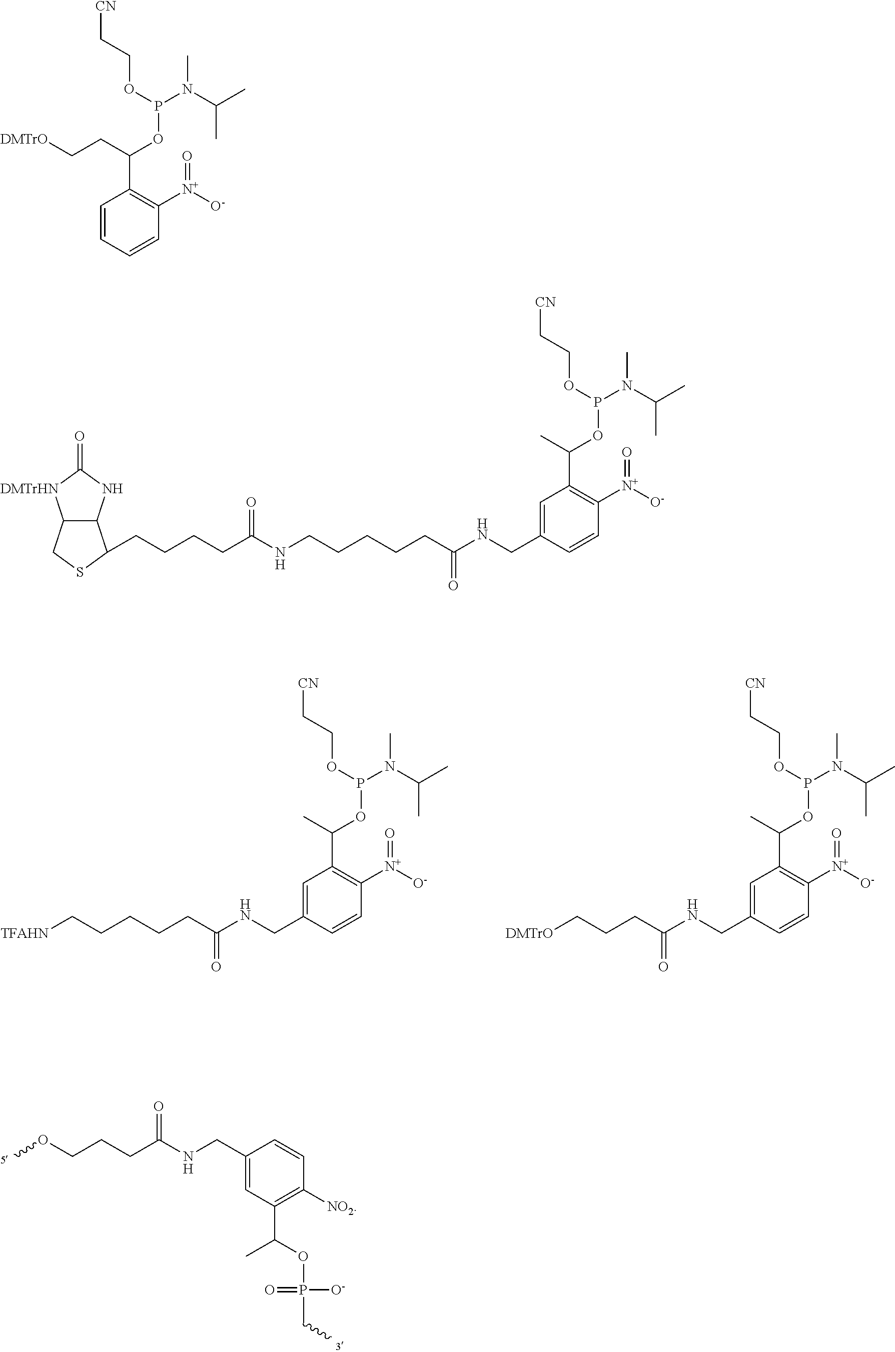

The above compounds are available commercially, and may result in a 5′-phosphate upon photocleaving, which can be used for subsequent reactions.

Non-limiting examples of photocleavable protection groups include:

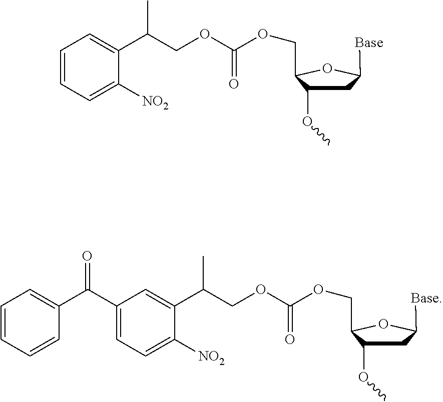

The above compounds are available commercially, and result in a 5′-OH upon photocleaving. The 5′-OH can be phosphorylated using for example, T4 polynucleotide kinase, to generate a 5′-phosphate, which can be used for subsequent ligation reactions.

In one set of embodiments, a first nucleic acid containing a photocleavable linker may be exposed to light, e.g., UV light, to expose a 5′-phosphate. The 5′-phosphate can then be used for attaching a second nucleic acid, e.g., using a DNA ligase (e.g., T4 DNA ligase). Other examples of DNA ligases include DNA ligase I, II, III, or IV, E. coli DNA ligase, etc.

In addition, in some cases, the first nucleic acid may also contain a spacer sequence. The spacer sequence may comprise, for example, natural or modified nucleotides or other molecules that can inhibit the attachment of nucleic acids, e.g., by DNA ligase. However, in certain embodiments, the photocleavable linker can be cleaved by exposure to light, which may result in the removal of the spacer sequence and exposure to the 5′-phosphate. Thus, by cleaving the photocleavable linker, the first nucleic acid can be used for the subsequent attachment of a second nucleic acid, e.g., at the 5′-phosphate.

In certain embodiments, when light (e.g., ultraviolet light) is applied, the photocleavable linker may be cleaved, thereby allowing the spacer sequence to leave. This may permit additional nucleic acids to be attached to the DNA tag or other nucleic acid. In some cases, the photocleavable linker may be one which, when reacted by light, causes the exposure of a 5′ phosphate group, which can facilitate the attachment of additional nucleic acids.

In another set of embodiments, a first nucleic acid containing a photocleavable protection group may be exposed to light, e.g., UV light, to expose a 5′-OH. The 5′-OH can then be phosphorylated using, for example, T4 polynucleotide kinase, to generate a 5′-phosphate. The 5′-phosphate can then be used for attaching a second nucleic acid, e.g., using a DNA ligase (e.g., T4 DNA ligase). Other examples of DNA ligases include DNA ligase I, II, III, or IV, E. coli DNA ligase, etc.

In certain embodiments, the second nucleic acid may itself contain a second photocleavable linker or protection group, which may independently be the same or different than the first photocleavable linker or protection group. The second nucleic acid may itself also contain a second spacer sequence, which may be independently be the same or different from the first spacer sequence (if present). Thus, in some cases, the second photocleavable linker or protection group may be removed, e.g., by applying light. In some cases, this may generate a 5′phosphate or a 5-OH, which can be phosphorylated under certain conditions. This second 5′-phosphate can then be used for attaching a third nucleic acid in some embodiments.

This process may be repeated for any suitable number times or rounds. For example, this process may be repeated at least 2, at least 3, at least 4, at least 5, at least 7, at least 10, at least 15, at least 20, at least 25, at least 30, at least 50, at least 75, at least 100, at least 150, at least 200, at least 300, at least 400, at least 500, at least 700, or at least 1000, or more times. In this way, a plurality of nucleic acids may be controllably joined together, e.g., to produce a barcode.

In one set of embodiments, the attachment of nucleic acids may be facilitated through the use of splints. The splint may have, for example, a first portion and a second portion, where the first portion may bind to at least a portion of a first nucleic acid and the second portion sequence may bind to at least a portion of a second nucleic acid. For example, in some embodiments, a mixture of splints containing some or all possible combinations of complements to the previous DNA letters or other nucleic acids can then be applied. In some cases, this may include an overhang corresponding to the complement of the next letter. Some or all of the splints that match a DNA letter or other nucleic acid may be annealed onto the previous DNA letters or other nucleic acids. The next DNA letter or other nucleic acid, which may be linked to a spacer sequence with a photocleavable linker, can be ligated on using, for example, T4 DNA ligase, or other suitable ligases, e.g., as discussed herein.

In some embodiments, a splint oligonucleotide may be blocked with a blocking group at a 3′ end (for example, by a C3 spacer), e.g., to prevent or inhibit it from ligating onto the oligonucleotides. The splints can be detached, for example, using formamide or other suitable techniques, and the splints can be washed away (e.g., using saline, etc. as described herein), to make the system ready for the next round of nucleic acid (or DNA letter) addition. This process can be repeated in certain cases to continue to add DNA letters or other nucleic acids, e.g., to form a barcode.

Such methods of barcode creation can be understood from a recursive algorithm in some cases. A non-limiting example is depicted in FIG. 5. This example starts from at least DNA letters, Bᵢ and Bⱼ, already ligated onto the sample, with the DNA letter Bⱼ having a photocleavable spacer. In the spatial locations where the next DNA letter would be added, light (e.g. UV light) is applied to cause cleavage of the spacer sequence and expose a 5′ phosphate group. This would make the oligonucleotides at those locations ready for ligation. In other locations that were not photocleaved, there would be no 5′ phosphate group at the 5′ end of the oligonucleotides, and those oligonucleotides would not participate in ligation. Accordingly, by controlling where light is applied, different nucleic acids can be added to different portions of a sample.

The DNA “letters” or other nucleic acid sequences may be of any length. If more than one DNA letter is present, the DNA letters may each independently have the same or different lengths. A DNA letter may be thought of as encoding a unit of information. In some embodiments, multiple DNA letters may be combined together to form a barcode, e.g., that encodes information, much as a word may be composed of multiple letters.

Thus, if more than one DNA letter or other nucleic acids is used, e.g., to form a barcode, the DNA letters or other nucleic acids that are used may each independently have the same or different lengths. For instance, the DNA letter or other nucleic acid may be at least 1, at least 2, at least 3, at least 4, at least 5, at least 6, at least 7, at least 8, at least 9, at least 10, at least 12, at least 15, at least 20, at least 25, at least 30, at least 35, at least 40, at least 50, at least 60, at least 65, at least 70, at least 75, at least 100, at least 200, at least 300, at least 400, at least 500, at least 600, at least 700, at least 800, at least 900, or at least 1,000 nucleotides in length. In some cases, the DNA letter or other nucleic acid may be no more than 1,000, no more than 900, no more than 800, no more than 700, no more than 600, no more than 500, no more than 400, no more than 300, no more than 200, no more than 100, no more than 75, no more than 70, no more than 65, no more than 60, no more than 50, no more than 40, no more than 35, no more than 30, no more than 25, no more than 20, no more than 15, no more than 12, no more than 10, no more than 9, no more than 8, no more than 7, no more than 6, no more than 5, no more than 4, no more than 3, or no more than 2 nucleotides in length. Combinations of any of these are also possible, e.g., a DNA letter may have a length of between 10 and 30 nucleotides, between 5 and 8 nucleotides, between 5 and 50 nucleotides, between 10 and 20 nucleotides, between 4 and 9 nucleotides, between 10 and 30 nucleotides, between 300 and 500 nucleotides, etc.

In addition, a population of barcodes may have any suitable number of DNA letters or nucleic acid sequences that defines the population of barcodes. For example, a population of barcodes may use 1, 2, 3, 4, 5, 6, 7, 8, 9, 10, 11, 12, 13, 14, 15, 16, 17, 18, 19, 20, etc. DNA letters or nucleic acid sequences. More than 20 are also possible in some embodiments. In addition, in some embodiments, the population of barcodes may be formed from at least 1, at least 2, at least 3, at least 4, at least 5, at least 6, at least 7, at least 8, at least 9, at least 10, at least 11, at least 12, at least 13, at least 14, at least 15, at least 16, at least 20, at least 24, at least 32, at least 40, at least 50, at least 60, at least 64, at least 80, at least 100 DNA letters or nucleic acid sequences. In certain cases, no more than 100, no more than 80, no more than 64, no more than 60, no more than 50, no more than 40, no more than 32, no more than 24, no more than 20, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than one DNA letters or nucleic acid sequences may be present in a population of barcodes. Combinations of any of these are also possible, e.g., a population of barcodes may comprise between 10 and 15, between 7 and 10, between 60 and 100, between 20 and 40, etc. DNA letters or nucleic acid sequences in total. Additionally, the barcodes in a population of barcodes may each independently have the same or different numbers of DNA letters or other nucleic acid sequences.

As discussed herein, the attachment of such DNA letters or other nucleic acids may be controlled, e.g., via light, as discussed herein. In some embodiments, this may be advantageously used to create unique nucleic acid sequences or “barcodes” that encode certain types of information, such as their spatial location, type of cell they are in, an experiment index, an image, a text document, or the like. In some cases, the barcodes may encode any arbitrary data, e.g., similar to a hard drive or other computer memory device. In addition, in certain embodiments, the barcodes may include error-detecting and/or error-correcting codes.

In some embodiments, a first DNA letter or other nucleic acid may be added by using reactions which are controlled by light. The light may be applied to a sample, e.g., to one or more locations of the sample. In some cases, beams of light may be directed or focused onto specific locations of a sample, while other locations of the sample are not exposed to such beams of light. Instead, such locations may be left in the dark, or at least be illuminated only by incidental light, e.g., light not specifically directed at those locations. There may be one or more than one beam of light directed at a sample at specific points in time.

For example, in one set of embodiments, the beams of light may be used to remove a protection group from a DNA tag or other nucleic acid, which may allow the DNA tag or other nucleic acid, which may allow additional nucleic acids to attach to it, e.g., via a ligation or other suitable reaction. The protection group, when present, may inhibit the attachment of nucleic acids using DNA-binding enzymes, for example, DNA polymerases (e.g., TdT), DNA ligases (e.g., T4 DNA ligase), or the like.

As a schematic non-limiting example, to administer the first DNA letter or other nucleic acid (e.g., “B₁”), all locations that are encoded with B₁ as the first DNA letter (or other nucleic acid) can be illuminated with light in order to add that DNA letter (or other nucleic acid) to the sample. This can be repeated for locations that use B₂ as the first DNA letter, and so on until Bn. This completes the synthesis of the first DNA letter (or other nucleic acid) onto desired molecules within the sample, and this process can then be repeated to add the second DNA letter to the barcode, etc. until as many DNA letters are added as desired.

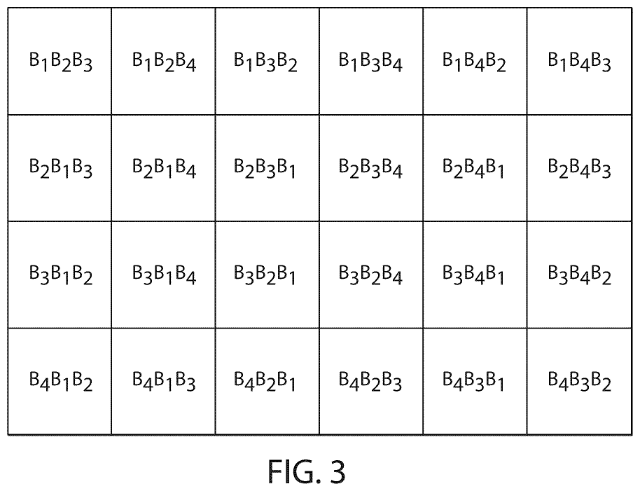

As an example, for m DNA letters in a barcode, the total number or DNA letter additions can be m×n or lower (for example, for the example shown in FIG. 3, three such rounds with 4 additions per round could be used since each barcode is formed from 3 unique DNA letters and 4 different DNA letter possibilities). In such a way, a combinatorically large number of barcodes can be generated from a much smaller number of DNA letter (or other nucleic acid) addition, in certain embodiments.

As a non-limiting example of an approach to combinatorically identifying a relatively large number of barcodes from a relatively small number of DNA letters (or other nucleic acid sequences) additions, a population of 4 different DNA letters is now described. It should be understood that although 4 DNA letters are used in this example for ease of explanation, in other embodiments, larger numbers of barcodes may be realized, such as discussed herein, for example, by using 5, 8, 10, 16, 32, etc. or more different DNA letters, or any other suitable number of DNA letters such as is described herein, depending on the application.

As a non-limiting example, if each of the barcodes contain two different DNA letters, then by using 4 such DNA letters (A, B, C, and D), up to 6 barcodes may be used if ordering is not essential and repeats are forbidden (AB, AC, AD, BC, BD, CD), or up to 16 if ordering is essential and repeats are required (AA, AB, AC, AD, BA, BB, BC, BD, CA, CB, CC, CD, DA, DB, DC, DD). This can be increased even further if the barcodes need not all contain the same number of DNA letters; for example, up to 20 could be obtained in this illustrative example (A, AA, AB, AC, AD, B, BA, BB, BC, BD, C, CA, CB, CC, CD, D, DA, DB, DC, DD). In all of these cases, the barcode can be generated by 2 rounds×4 letters=8 round of DNA letter addition.

As another non-limiting example of using letters with a combination of bases, the DNA letters are among these 6 different 5 base long sequences: AGAGA, ATGGA, TAGGT, TGTGT, AAGGT, TTGGA. A barcode of one DNA letter can have one of the six possibilities. A barcode with two DNA letters can have 6 options for the first letter and 6 options for the second letter for a total of 6² total possibilities, and can be used to produce sequences such as

(SEQ ID NO: 45)
AGAGA AGAGA

or

(SEQ ID NO: 46)
AAGGT TTGGA,,

etc. For a barcode with three DNA letters, there can be up to 6³ possibilities, for example, with sequences that look like

(SEQ ID NO: 47)
AGAGA TGTGT AGAGA

or

(SEQ ID NO: 48)
TGTGT TGTGT TTGGA,

etc. For a barcode with three DNA letters, there would be 6^{m} possibilities, which grows exponentially with number of DNA letters. The number of additions would be 6×m and only scale linearly with the number of DNA letters. For m=3 this would be 216 barcodes in 18 additions or for m=10, this would be 60466176 barcodes in 60 additions.

As another non-limiting example of using letters with a combination of bases, the DNA letters are among these 6 different 5 base long sequences: AGAGA, ATGGA, TAGGT, TGTGT, AAGGT, TTGGA. In this example, in addition, a DNA letter may be chose to not be repeated with the previous two DNA letters. A barcode of one DNA letter can have one of the six possibilities. A barcode with two DNA letters can have 6×5 possibilities, since there cannot be a repeat with the previous DNA letter, and this can be used to produce sequences such as

(SEQ ID NO: 49)
AGAGA ATGGA

or

(SEQ ID NO: 50)
TAGGT TTGGA,

etc. For a barcode with three DNA letters, there can be up to 6×5×4 possibilities, e.g., with sequences such as

(SEQ ID NO: 51)
AGAGA TGTGT ATGGA

or

(SEQ ID NO: 52)
TGTGT ATGGA TTGGA,

etc. For a barcode with four DNA letters, there can be up to 6×5×4² possibilities, with sequences such as

(SEQ ID NO: 53)
AGAGA TGTGT ATGGA AGAGA.

In some cases, some of the DNA letters may repeat and not be the same as the previous two DNA letters. As the barcode is expanded to m DNA letters, there would be up to 6×5×4^{(m-2)} possibilities, which may grow exponentially with number of DNA letters. The number of additions are 6+5+4×(m−2) and also grows linearly with the number of DNA letters.

It should be understood that although the above examples used only 6 different 5 base long DNA letters, for ease of understanding, in other embodiments, DNA “letters” or other sequences with more or less than 5 nucleotides can be used as well, which may be combined to form barcodes, e.g., of any suitable length. For example, a barcode may comprise at least 1, at least 2, at least 3, at least 4, at least 5, at least 6, at least 7, at least 8, at least 9, at least 10, at least 12, at least 15, at least 20, at least 25, at least 30, at least 35, at least 40, at least 50, at least 60, at least 65, at least 70, at least 75, at least 100, at least 200, at least 300, at least 400, at least 500, at least 600, at least 700, at least 800, at least 900, or at least 1,000 DNA letters (or other sequences).

As these non-limiting examples illustrate, a relatively large number of barcodes may be formed or synthesized within a sample in certain embodiments, e.g., using techniques such as those described herein, based on a relatively small number of DNA letters or other nucleic acid sequences. Using spatial patterning of light this also correspondingly results in a small number of DNA letter additions. Such barcodes may be used, for instance, to encode certain types of information, such as their spatial location, and/or other types of information. For example, a barcode may encode information about the experiment number, the applied experimental conditions, reaction conditions used, even abstract data such as text, or the like. In some cases, the barcodes may encode any arbitrary data, e.g., similar to a hard drive or other computer memory device. In addition in some cases, a barcode may encode more than one type of information, for example, spatial location and experiment number.

In addition, certain aspects are generally directed to forming or synthesizing nucleic acids at particular locations within a sample, e.g., at specific spatial locations within the sample. These may be attached to DNA tags or other nucleic acids contained within the sample, and/or to other targets in certain cases. A variety of reactions may be used to attach DNA letters or other nucleic acids to a sample, such as those discussed herein. Examples of such techniques include those described in U.S. Provisional Patent Application Ser. No. 63/458,610, filed Apr. 11, 2023, entitled “Multiplexed Optical Barcoding for Spatial Omics,” by Zhuang, et al., and a patent application filed on even date herewith, entitled “Multiplexed Optical Barcoding for Spatial Omics,” by Zhuang, et al., each incorporated herein by reference in its entirety.

Thus, by using reactions such as these, certain aspects as discussed herein may be generally directed to systems and methods for controlling the attachment of DNA “letters” or nucleic acids sequences to molecules such as DNA tags or other nucleic acids that may be present within a sample. A sample may include a cell culture, a suspension of cells, a biological tissue, a biopsy, an organism, a slide surface, or the like. The sample can also be cell-free but nevertheless contain nucleic acids in some cases. If the sample contains a cell, the cell may be a human cell, or any other suitable cell, e.g., a mammalian cell, a fish cell, an insect cell, a plant cell, or the like. More than one cell may be present in some cases.

In some embodiments, cells, tissues, or other samples may be permeabilized, e.g., to allow such fluid flow to occur. In certain embodiments, components within a cell, tissue, or other sample may be fixed. Those of ordinary skill in the art will be familiar with techniques for permeabilizing or fixing cells or other samples. If the sample is a slide surface, for example, DNA tags or other nucleic acids may be covalently or non-covalently attached to the surface to fix them in place, for example, using a biotin modified oligonucleotide attached to a streptavidin coated surface, covalently through an amine modified oligonucleotide on a surface coated with NHS, or other techniques for attaching nucleic acids to a surface. As another example, the sample can also be a test tube with free floating oligonucleotides.

In addition, in some embodiments, DNA tags or other nucleic acids may be attached or immobilized, e.g., on a surface, using a chemically cleavable linker such as succinyl linker. The DNA tags with barcodes may then be extracted or removed, in some embodiments, using ammonium hydroxide and then sequenced, etc., as discussed herein.

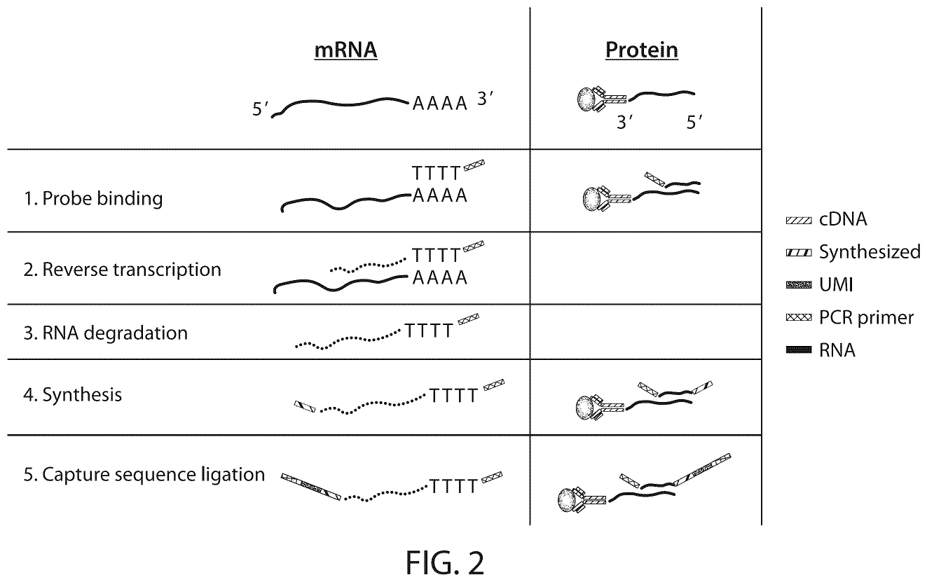

FIG. 2 illustrates one non-limiting example for introducing DNA tags or other nucleic acids into a sample. In this figure, DNA tags are attached to mRNAs by using the 3′ end poly-A of the mRNAs to attach a poly-T DNA tag onto it. Reverse transcription may then be used to copy the mRNA content onto the DNA tag. A variety of reverse transcriptases are commercially available. The RNA may then be digested, e.g., using RNAse H, leaving the copied DNA tag.

In addition, certain embodiments as discussed herein are generally directed to systems and methods for controlling the attachment of DNA letters or nucleic acids sequences to molecules such as DNA tags or other nucleic acids that may be present within a sample. As discussed herein, the placement of such DNA letters or other nucleic acids may be spatially controlled, e.g., by using the application of light, such that in locations where light is applied, the DNA letters or other nucleic acids are added to DNA tags or other nucleic acids within the sample, while in locations where light is not applied (or where the light is not specifically directed at those locations), the DNA letters or other nucleic acids may not be added to such DNA tags, nucleic acids, etc., within the sample. Thus, by controlling where light is applied to a sample, the locations where nucleic acids are added can be controlled, e.g., spatially. Thus, different nucleic acid sequences can be formed or synthesized at different spatial locations within a sample.

Accordingly, in certain embodiments, spatial information may be introduced with the barcodes. For example, in some embodiments, spatial locations within a sample may be discretized into pixels. The discretization of space may occur in 2 dimensions, or 3 dimensions in some instances, e.g., as discussed below. In addition, in certain embodiments, some or all spatial locations (e.g. pixel or voxel) may be identified, in some cases uniquely, with a specific sequence or barcode. As discussed below, within a spatial location, different barcodes may be defined. In some cases, such barcodes may be formed or synthesized, e.g., by attaching appropriate DNA letters or other nucleic acids to molecules such as DNA tags or other nucleic acids within that spatial location (e.g., if conditions are appropriate), for example, by controlling light such that it is applied to only those spatial locations to which appending or attachment of nucleic acids is desired. In some embodiments, a unique DNA letter or other nucleic acid sequence may be assigned to each spatial location, although in other embodiments, different spatial locations may be identified, for example, by using unique combinations of DNA letters, e.g., to form different barcodes identifying the different spatial locations. In various embodiments, DNA barcodes may be defined as various permutations or combinations of DNA letters or other nucleic acids, e.g., where the order matters or does not matter. Examples of methods for the light-directed enzymatic ligation steps, followed by the description of creating spatial light patterns, are discussed in more detail herein. The photocleavable linker or protection group can be cleaved using light, such as UV light, which is achievable using commercial LED or laser options, or other suitable light sources. Examples of spatial light modulators (SLMs) that may be used include, but are not limited to, liquid crystal on silicon (LCOS) chips, digital micromirror devices (DMDs), acousto-optic deflectors (AOD), etc.

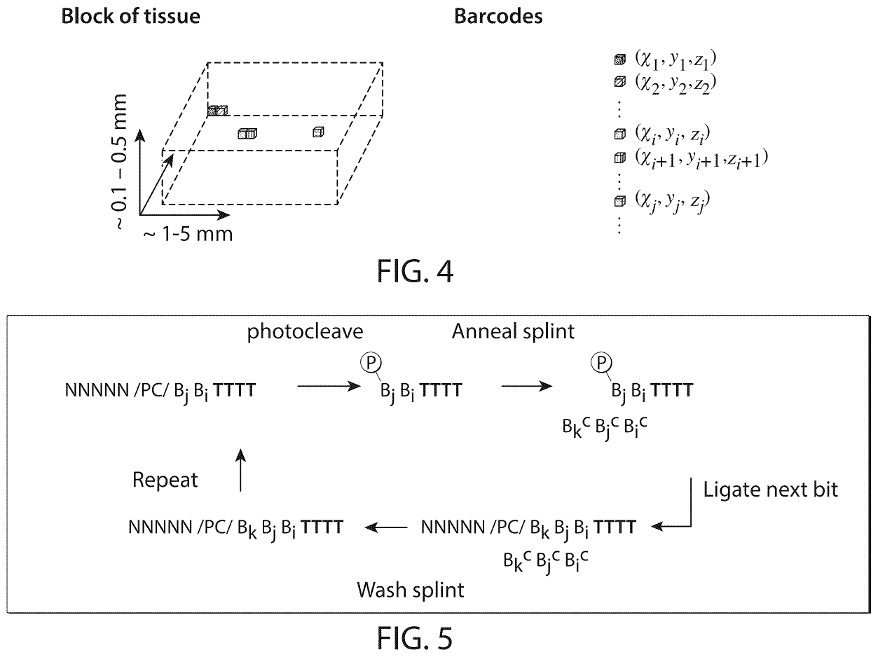

As a schematic non-limiting example, in FIG. 3, an example 2-dimensional region of space has been discretized, and each “pixel” uniquely identified with a barcode. The various possible DNA letters are represented as B₁, B₂, . . . , Bm and can include any suitable combination of bases, such as the four A, T, G, and C bases, and/or other non-naturally occurring bases in some cases. In addition, in some embodiments, some or all of the DNA letters may include more than one nucleotide, e.g., such as described herein. The DNA letters can be combined to form a barcode, for example, B₁B₂B₃, B₁B₂B₄, B₂B₁B₃, etc., as is shown in FIG. 4 with various combinations of B₁, B₂, B₃, and B₄ forming different barcodes. Other encoding techniques can also be used in other embodiments. For example, in some embodiments, the ordering may be important (e.g., in FIG. 3, B₁B₂B₃ and B₂B₁B₃ encode different spatial positions), while in other embodiments, ordering may not be important, and barcodes differentiated on the basis of content or concentrations and/or types of DNA letters or other nucleic acid sequences (as a non-limiting example, barcodes such as B₁B₂B₃ and B₂B₁B₃ may encode the same spatial position, while barcodes such as B₁B₁B₂ and B₁B₂B₂ may encode different spatial positions, e.g., due to the different concentrations of B's in each).

In some embodiments, various systems may allow for multiplex positional encoding in some cases, e.g., where unique combinations of DNA letters in a barcode may allow for different spatial positions to be uniquely identified, rather than using a unique DNA letter for each spatial location in other embodiments (however, in other embodiments, unique DNA letters may be used for each spatial location). Moreover, in some embodiments, the DNA letters may or may not be repeated within a barcode, e.g., in various applications.

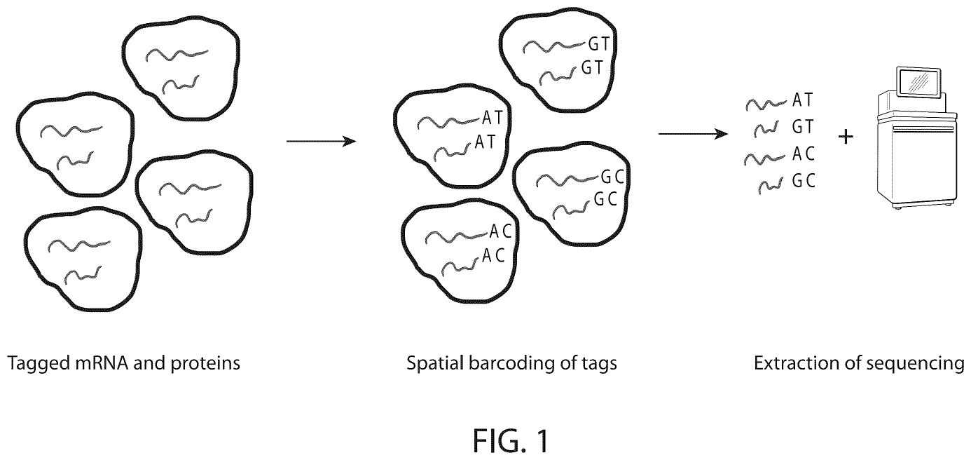

In one set of embodiments, nucleic acids (e.g., comprising DNA and/or RNA), such as DNA letters, may be added to a sample in spatially controlled positions by the application of light. Thus, different spatial locations within a sample may have attached to them different nucleic acids, which may allow the spatial locations to be uniquely identified. In some cases, the application of light may be controlled such that at certain spatial positions, multiple nucleic acid sequences can be added, e.g., to the sample, and/or to other nucleic acids such as DNA tags, for example, that may be present within the sample. The other nucleic acids within the sample may be endogenous to the sample, and/or may have been previously attached, e.g., in prior rounds of nucleic acid attachments, using these or other techniques. In this way, in accordance with certain embodiments, a “barcode” of DNA letters or other nucleic acids can be formed, e.g., by using the application of light (for example, as beams of light) to control the addition of various nucleic acids at those spatial positions. In contrast, in other locations of the sample, the nucleic acids and/or specific combinations of nucleic acids may not present. Thus, in some embodiments, various spatial positions within the sample can be determined based on the nucleic acids that are present. One schematic illustration of this process is shown in the example of FIG. 1.

As another example, ligation-based DNA barcoding can be used in various embodiments. For example, ligation of photocleavable oligonucleotides can be used to introduce spatial barcodes. In this case, some or all of the DNA letters or other nucleic acids in a barcode may comprise a sequence of nucleotides, which may be linked to a spacer sequence, e.g., by a photocleavable linker.

After the DNA letters are added, the barcode that they represent may uniquely map onto a physical position on the sample in some embodiments (e.g., as illustrated in the example shown in FIG. 4). In some cases, the same DNA letter may be applied in different rounds (e.g., when the same DNA letter is used in different positions within the barcode), although in other cases, the same DNA letter may not necessarily be applied in different rounds (e.g., when different DNA letter are used in different positions within the barcode).

As mentioned, in certain embodiments, arbitrary data, encoding text, images, or the like may be encoded in barcodes. One non-limiting example for encoding an image using barcodes is follows. In some embodiments, DNA tags or other nucleic acids may be attached to or immobilized on a surface. The surface may be discretized into pixels, and the DNA tags or nucleic acids on each pixel may be barcoded to encode their spatial information, e.g., as described in detail herein. The spatial barcoding may be done using spatially controlled light, or other techniques. In some cases, the arbitrary data (e.g., image data, or other data such as described herein) can be encoded using an integer between 0 and 255, which can converted into a barcode. (Other encoding schemes, e.g., using other integer values, are possible in other embodiments, although 256 was chosen in this example for ease of explanation, and because of its widespread use in computers). One method of encoding integer values between 0 and 256 can be achieved using 4 DNA letters (A, B, C, D) and a barcode that is 4 DNA letters long. (As discussed herein, the DNA letters may each be defined as one, or more, nucleotides). Combined, these DNA letters can form barcodes such as ABCD, ADCB, AABB, etc., totaling 4⁴=256 possibilities, which is the number of barcodes needed to encode integers 0 through 255. Accordingly, in this example, The arbitrary data (e.g., image data) can be encoded in a barcode, and the data barcode and the spatial location barcode can be combined to encode arbitrary data, for example, by creating a representation of an image using 0-255 intensities (e.g., a grayscale image), by encoding text, or the like.

Each of the following is incorporated herein by reference in its entirety: U.S. Provisional Patent Application Ser. No. 63/506,337, filed Jun. 5, 2023, entitled “Chemical Ligation Techniques,” by Zhuang, et al.; U.S. Provisional Patent Application Ser. No. 63/458,610, filed Apr. 11, 2023, entitled “Multiplexed Optical Barcoding for Spatial Omics,” by Zhuang, et al.; and U.S. Provisional Patent Application Ser. No. 63/506,294, filed Jun. 5, 2023, entitled “Multiplexed Optical Barcoding for Spatial Omics,” by Zhuang, et al.

The following examples are intended to illustrate certain embodiments of the present disclosure, but do not exemplify the full scope of the disclosure.

### Example 1

As a non-limiting example, an overhanging sequence that is 5 bases long and with at least 40% GC content has close to 100% ligation efficiency within 10 minutes. The melting temperature of splints that have two letters with 5 bases and 40% GC content is >25° C. and this example illustrates that these can be annealed during ligation and removed with a 30% formamide solution after the ligation is completed. Through this recursive mechanism, barcodes for spatial location multiplexing can be prepared.

The following example illustrates the approach of ligation and photocleaving in vitro in a test tube. A DNA tag

(P = /5Phos/ AGAGA TGTGT TGTGT T(10) /36-FAM/
(SEQ ID NO: 54))

that has a fluorophore in its 3′ end may be used. A DNA letter with a photocleavable spacer

(A = T(25) (SEQ ID NO: 55)/iSpPC/ AGAGA)

may be ligated using a splint

(S = ACACA TCTCT TCTCT (SEQ ID NO: 56))

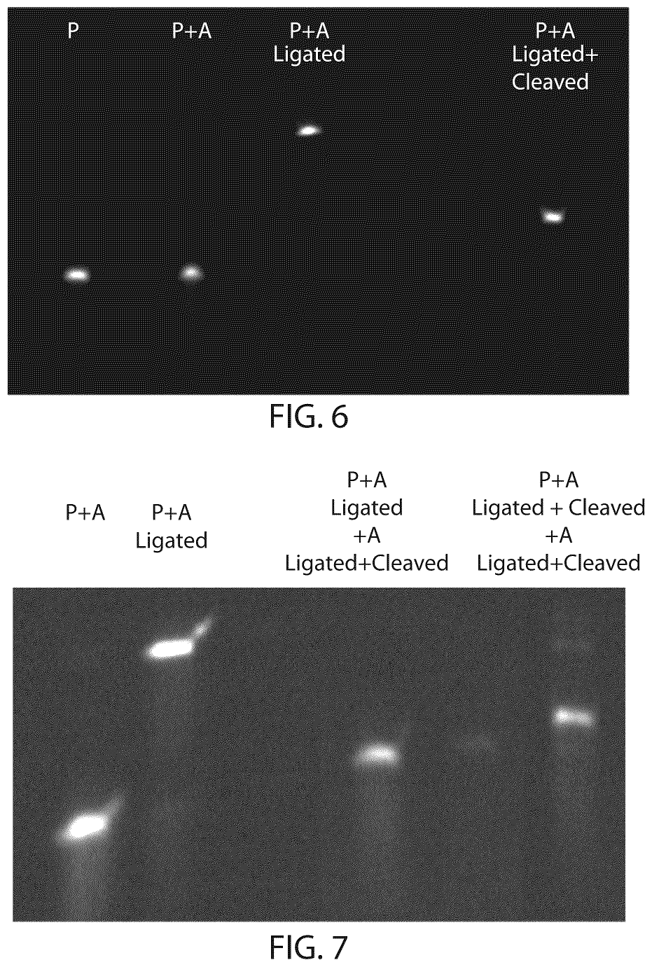

using Quick Ligation™ Kit (vendor: New England Biosciences) and the products of the reaction run on a 15% TBE (tris borate EDTA) urea denaturing gel. 3 pMol of the DNA tag, 30 pMol of the DNA letter, 30 pMol of the splint and 1 microliter of the Quick Ligase™ Enzyme were used in a 20 microliter reaction for 10 minutes at room temperature. The DNA tag, letter, and splints were ordered from IDT and diluted to 100 micromolar in nuclease free water and stored at −20° C. After 10 minutes, the reaction products were purified in 10 microliters of nuclease free water using Monarch DNA Cleanup kit from NEB before running on the gel. This assay allows for the quantification of the ligation and photocleaving efficiency directly by observing the location of the fluorescent bands on the gel. The results of this assay are shown in FIG. 6: column 1 is the primer, column 2 has the primer, letter, and splint but no ligase enzyme, column 3 is the ligated reaction for 5 minutes, column 4 is the ligated reaction followed by 10 minutes of photocleaving using a 365 nm LED at an intensity of 0.1 W/cm². From the band shifts in the gel, the ligation efficiency was estimated at greater than 95% and the photocleaving efficiency was estimated at greater than 95%.

### Example 2

The following example shows ligation of a DNA letter in a second iteration after photocleaving the previous DNA letter. The same DNA tag and DNA letter as Example 1 was used in this assay. The DNA letter was first ligated using splint S and photocleaved as above. For the next ligation reaction, a longer splint

(S2 = ACACA ACACA TCTCT TCTCT TCTCT
(SEQ ID NO: 57))

was used, which preferentially attaches to the primer over the shorter splint.

The products of the ligation reaction from Example 1 after photocleaving were purified into 3 microliters of nuclease free water using Monarch DNA Cleanup kit from NEB. 3 microliters of 20 micromolar splint (S2) and 3 microliters of 10 μM DNA letter were added along with 1 microliter Quick Ligase™ Enzyme in a 20 microliter reaction for 10 minutes at room temperature. After 10 minutes, the reaction products were purified in 10 microliter nuclease free water using Monarch DNA Cleanup kit from NEB before running on the gel.

FIG. 7 shows the results of this assay wherein column 1 is the unligated primer and letter, column 2 is the ligated primer and letter, column 3 is the second round of ligation without photocleaving the first round, column 4 is the second round of ligation after photocleaving the first round. From the size shifts, it can be estimated that the second ligation reaction also occurred with efficiency of greater than 95%. In addition, it was also shown that there was no further ligation reaction without photocleaving.

### Example 3

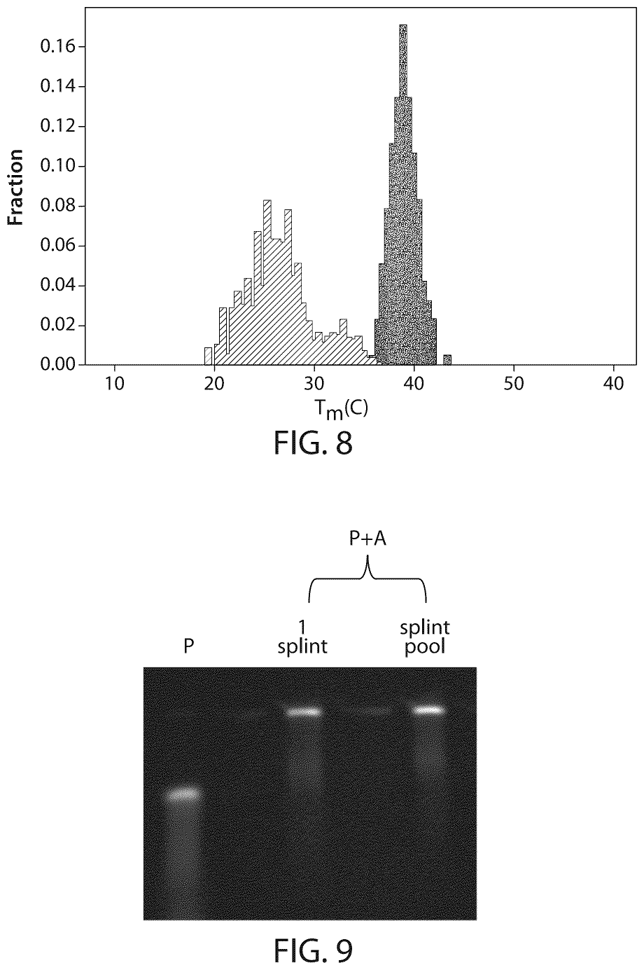

This example illustrates that a pool of splints can be used in order to have spatial multiplexing as discussed in the above examples. The first step in this process was to design a set of letters and splints allowing the addition of the next letter efficiently when a pool of splints is present. Simulations were run over various choices letters containing 5 bases and a 40% GC content such that the melting temperature of a splint with the correct complementary sequence (right histogram in FIG. 8) is distinct from the melting temperature of a splint with one letter mismatch (left histogram).

The results are shown in FIG. 8 where the melting temperature of the wrongly matched splints are centered at room temperature and would constantly detach off the primer until the correct splint, whose melting temperature is much higher than room temperature, anneals on. The ligation assay was repeated as before using DNA tag

(P = /5Phos/TGTGT TAGGT ATGGA AGAGA T(10)/36-FAM/)
(SEQ ID NO: 58))

and DNA letter

(A = T(25) (SEQ ID NO: 55)/iSpPC/AGAGA)

using either a splint with correct complementarity or a splint with all possible prior letter combinations. The concentration and reaction conditions are the same as Example 1. In the case of the splint pool, 3 microliters of 10 micromolar concentration per splint was used. The reaction products were column purified as before and run on a 15% TBE (tris borate EDTA) urea denaturing gel. The results are shown in FIG. 9 and shows that the ligation efficiency was just as good (greater than 95%) using a splint pool.

### Example 4

The above reactions were tested in-vivo in cultured U2OS cells in this example. The cells were plated onto Ibidi μ-Slide VI 0.4 fluidics chamber with ibiTreat coating and cultured overnight. The cells were fixed for 10 minutes with 4% PFA and permeabilized with 0.25% Triton X-100 in PBS for 10 minutes. The sample was then washed with PBS. A DNA Tag

(SEQ ID NO: 59)
(P = /5Phos/ TGTGT TAGGT ATGGA AGAGA T(45)

R2, R2 is a PCR primer) is annealed onto the poly-A tail of RNA molecules inside cells using the following buffer: 2 microliters of 25% Triton X-100, 1 microliter of 100 micromolar DNA Tag, 6 microliters of RNAse inhibitor, 20 microliters of 5×RT reaction buffer, and 71 microliters of H₂O. A DNA letter

(A1 = T(25) (SEQ ID NO: 55)/iSpPC/AGAGA)

was ligated using the splint pool from example 3 and photocleaved. 1 microliter of 50 micromolar A1, 1 microliter of 50 micromolar per splint in splint pool, 1 microliter of Quick Ligase™ Enzyme, 22 microliters of nuclease free water in a 50 microliter reaction was used, followed by incubation for 10 minutes at room temperature. The chamber was washed twice with a 0.2×SSC, 50% formamide solution and then with PBS. The sample was photocleaved for 10 minutes using a 365 nm LED at an intensity of 0.1 W/cm². Another DNA tag

(A2 = T(25) (SEQ ID NO: 55)/iSpPC/ATGGA),

was ligated using the corresponding splint pool and photocleaved as in the previous round. Finally a PCR primer (R1) was ligated as in the previous rounds. The oligonucleotides were extracted by degrading the RNA using RNAseH (1.5 microliters RnaseH from NEB in thermopol buffer and 50 microliters reaction volume). The extracted oligonucleotides were PCR amplified and run on Invitrogen™ E-Gel™ EX Agarose Gels, 4% to determine size. Only the extracted oligonucleotides with the PCR primer R1 were amplified.

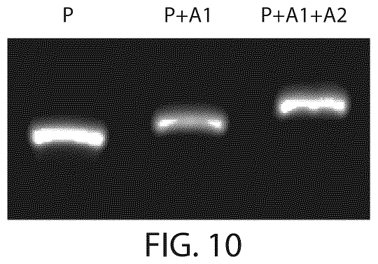

Suppose the efficiency of in-vivo ligation is el and the combined efficiency of photocleaving and washing is ew. In the assay where only A1 was ligated with R1, the intensity ratio of the gel band corresponding to one letter addition A1 compared to no letter addition would be (el*ew)/(1−el). In the assay where A1, A2, and R1 were ligated, the intensity ratio of the gel band corresponding to two letter addition compared to one letter addition would be (el*ew)/(2−el−ew). This allows for the determination of el and ew from FIG. 10 in vivo in cultured cells to be greater than 98% each.

### Example 5

This example demonstrates>95% efficiency per photosensitive ligation over 4 ligations in cultured U2OS cells. The cells were plated in a Ibidi μ-Slide VI 0.4 coated with poly-d-lysine and grown overnight. The cells were fixed for 10 minutes with 4% PFA and washed with 1×PBS. A DNA tag

(SEQ ID NO: 60))
(/5Phos/AGAGA ATGGA TAGGT TGTGT
AATCAGCCATACCACATTTG TT +TT +TT +TT +TT +TT
+TT +TT +TT +TT +TT +TTT

was hybridized onto the poly-A tail of RNA molecules inside the cells/tissue at 1 μM concentration in 2×SSC at 37 C overnight. The samples were washed two times with 30% formamide in 2×SSC at 47 C for 30 mins to remove any unspecifically bound DNA tag. 4 different DNA letters were used

(L1= T(25) (SEQ ID NO: 55)/iSpPC/
AGAGA ATGGA TAGGT TGTGT (SEQ ID NO: 61),
L2 = T(25) (SEQ ID NO: 55)/iSpPC/
ATGGA AGAGA TGTGT TAGGT (SEQ ID NO: 62),
L3 = T(25) (SEQ ID NO: 55)/iSpPC/
TAGGT TGTGT AGAGA ATGGA (SEQ ID NO: 63),
L4 = T(25)(SEQ ID NO: 55) /iSpPC/
TGTGT TAGGT ATGGA AGAGA (SEQ ID NO: 64)).

Letter L2 is first ligated onto the DNA tag using a splint

(S2 = TCCAT TCTCT ACCTA (SEQ ID NO: 21))

in the following buffer: 50 μl 2× ligation buffer, 42 μl H2O, 2 μl 40 μM letter, 2 μl 40 μM splint, 2 μl 25% TX-100, 2 μl Quick Ligase, where the 2× ligation buffer was made fresh with 132 mM Tris, 20 mM MgCl2, 2 mM ATP, 2 mM DTT, 15% PEG 8000 adjusted to pH 7.6 with 1M HCl. The ligation reaction was carried out at room temperature for 1 hour. The sample was photocleaved for 1 minute using a 365 nm LED at 0.1 W/cm². The sample was subsequently ligated with L1 using a splint

(S1 = TCTCT TCCAT ACACA (SEQ ID NO: 29)).

The sample was photocleaved again and ligated with L3 using a splint

(S3 = TCCAT TCTCT TCCAT (SEQ ID NO: 65)).

The sample was photocleaved again and ligated with L4 using a splint

(S4 = ACACA ACCTA TCTCT (SEQ ID NO: 12)).

The samples were then heated to 93 C for 3 minutes in water and the water containing the DNA tags was aspirated out. The solution was annealed with the sequence complementary to

(SEQ ID NO: 66)
AATCAGCCATACCACATTTG

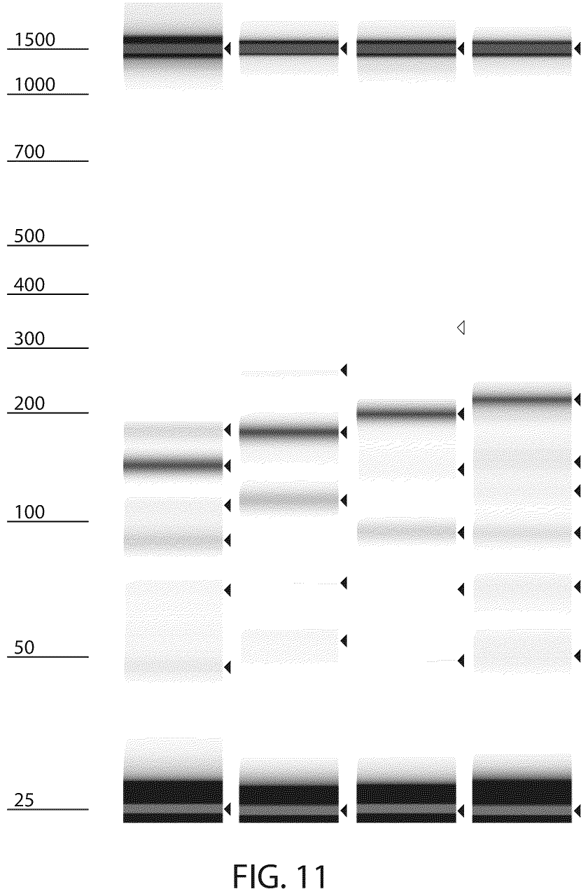

in the DNA tag and run on the HS DNA 1000 tape on an Agilent 4200 Tapesatation. The size observed on the tapestation is reflective of the DNA letters being ligated onto the tags and the efficiency of the ligations and photocleaving can be estimated from the relative height of the correct peak to all the other peaks. FIG. 11 shows the tapestation results for 1, 2, 3, and 4 ligations, with >95% efficiency per ligation.

### Example 6

This example demonstrates spatial selectivity of photocleaving using a Digital Mirror Device (DMD), wherein a fluorescent signal was used that only appears in the region that was photocleaved. Cultured cells were used, prepared as in the previous example, using the same DNA tag and ligated first with L2. Polygon1000-G from Mightex was used in a Olympus IX71 microscope body with a Nikon 10× plan apo λ objective and a region of 1 mm×0.6 mm was illuminated at 365 nm LED at 0.1 W/cm² for 1 minute. A letter

(L1-stv = GAT
CCG ATT GGA ACC GTC CC (SEQ ID NO: 67)/iSpPC/
AGAGA ATGGA TAGGT TGTGT (SEQ ID NO: 61))

was then ligated and the presence of L1-stv was detected using a fluorescently tagged conjugate of the sequence

(SEQ ID NO: 67)
GAT CCG ATT GGA ACC GTC CC

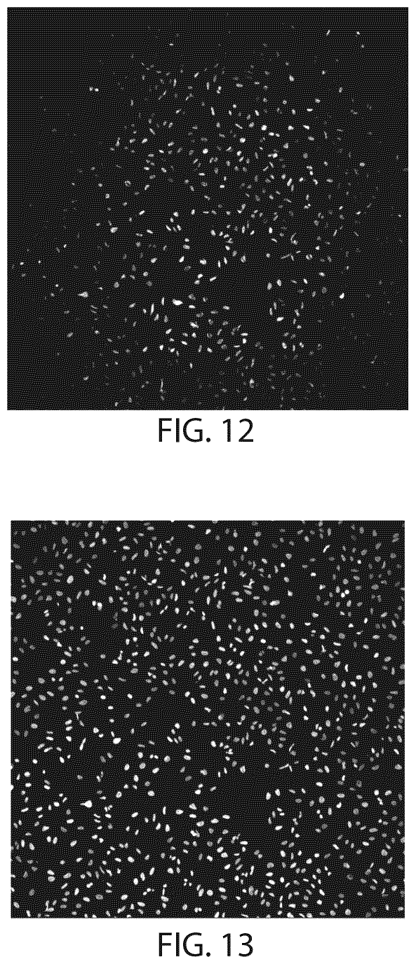

in L1-stv. FIG. 12 shows the fluorescent signal in the photocleaved region while FIG. 13 is a DAPI signal showing the presence of cells everywhere.

### Example 7

This example demonstrates that the mRNA content of tissue samples can be reliably captured using in-situ Reverse Transcription (RT), including with the ligation chemistry disclosed herein. To demonstrate this, Mouse brain cortex tissue was used to prepare three samples. In one of the sections, RNA was extracted and a sequencing library was prepared using SMART-Seq v4 3′ DE Kit. In another section, it was hybridized with DNA tag

(SS3 = ACGAGCATCAGCAGCATACGA
NNNNNNNNNNNNNNNNNNNN
TTTTTTTTTTTTTTTTTTTTTTTTTTTTTT
VN (SEQ ID NO: 68))

and in another section it was hybridized with DNA tag

(Lig = /5Phos/AGAGA ATGGA TAGGT TGTGT
NNNNNNNNNNNNNNNNNNNN
TTTTTTTTTTTTTTTTTTTTTTTTTTTTTTVN (SEQ ID NO: 69)).

After overnight hybridization, the samples were washed twice with 2×SSC and briefly with water and put into RT buffer (7.5 μl 25 mM dNTP, 25 μl 5×RT buffer, 1.5 μl RnaseIn, 1.5 μl Rnase inhibitor 40 U/μl, 2.5 μl 100 μM TSO, 5 μl Maxima H minus 200 U/μl, and 57 μl H2O, where the TSO was

/5Biosg/AAGCAGTGGTATCAACGCAGAGTACATrGrG+G
(SEQ ID NO: 70)).

The samples were incubated at 37 C overnight. The following day, the samples were washed twice with 2×SSC and twice with 1×PBS. For the sample with DNA tag Lig, the sequence

(SEQ ID NO: 73)
ACGAGCATCAGCAGCATACGA

was ligated. cDNA from sample was extracted in 1× Seqamp CB buffer with 1.5 μl of Rnase H in 100 μl total volume at 37 C for 1 hour. The extracted samples were PCR for 8 cycles and then tagmented using the Illumine Nextera XT kit and 5′ enriched using the PCR primers

(SEQ ID NO: 71)
GTCTCGTGGGCTCGGAGATGTGTATAAGAGACAG
and
(SEQ ID NO: 72)
TCGTCGGCAGCGTCAGATGTGTATAAGAGACAG
ACGAGCATCAGCAGCATACGA.

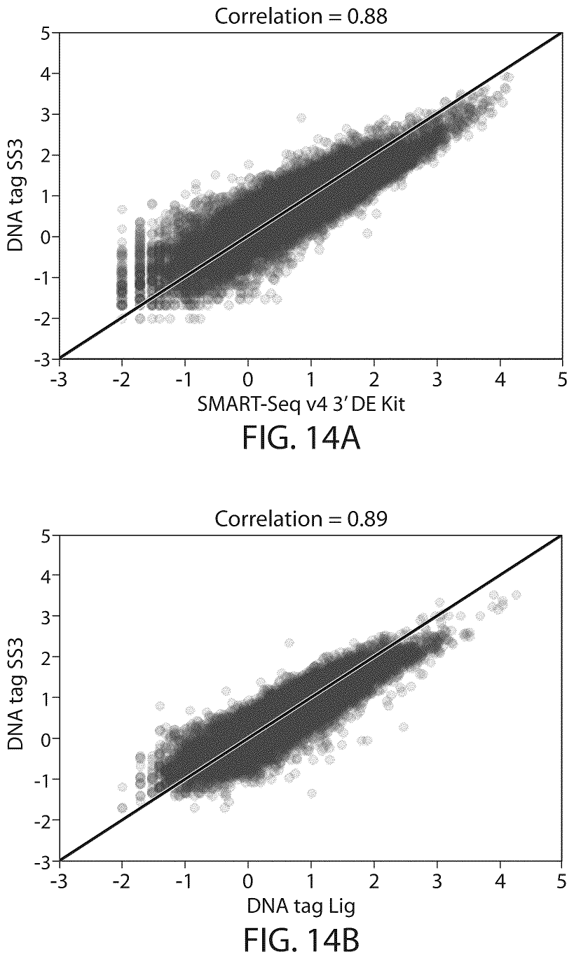

The samples were indexed and then sequenced at Novogene with 20 million reads each. Results are shown in FIGS. 14A-14B.

### Example 8

This example demonstrates the spatial selectivity of barcoding and validating it through sequencing. Two different species of cells, U2OS and MEF were plated next to each other in a Ibidi Culture-Insert 4 Well in μ-Dish coated with Poly-d-lysine. The cells were fixed and hybridized with primer

(Lig = /5Phos/AGAGA ATGGA TAGGT TGTGT
NNNNNNNNNNNNNNNNNNNN
TTTTTTTTTTTTTTTTTTTTTTTTTTTTTTVN (SEQ ID NO: 69).

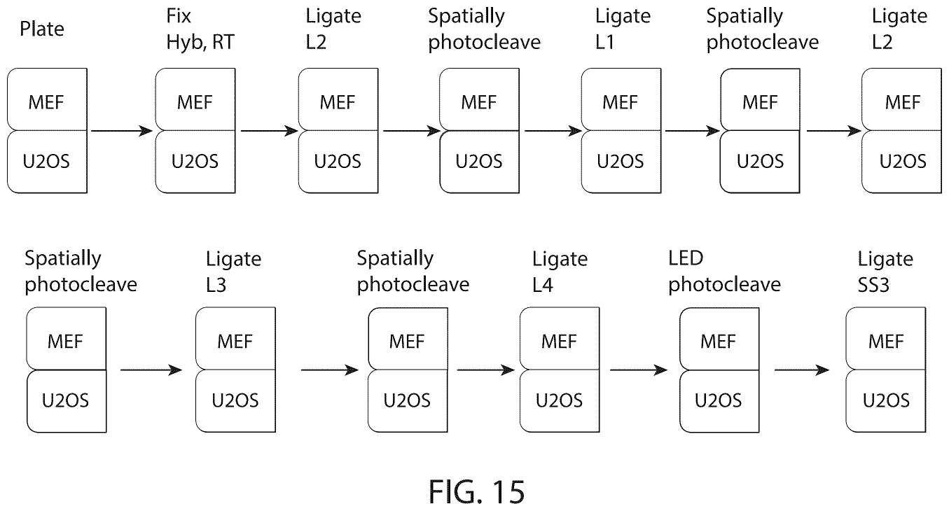

as described in Example 7. The sample was also reverse transcribed as above. After RT, in this case, the sample was washed twice in 30% formamide 2×SSC at 47 C for 30 mins. After the washes, the divider silicone chamber was removed and the ligation order in FIG. 15 was followed. SS3 was the PCR primer

(SEQ ID NO: 73)
ACGAGCATCAGCAGCATACGA

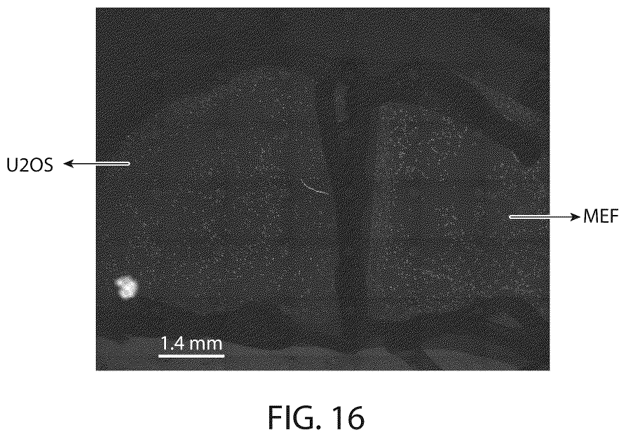

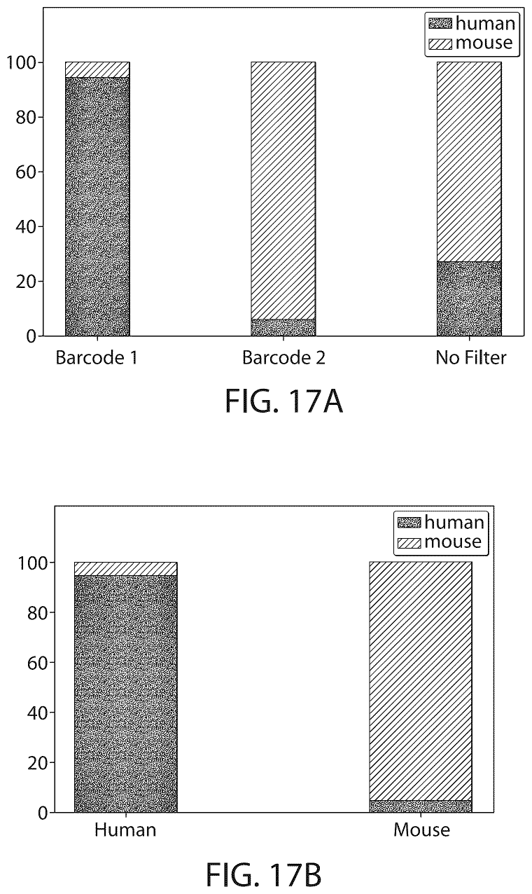

as in Example 7 and L1, L2, L3, L4 are the same letters as in Example 5. Each sample had two letters ligated in order to build in error-correction and improve the position identification efficiency. FIG. 16 shows a DAPI image of the cells. After the ligations, the cDNA with the DNA tag and barcode was extracted in water at 93 C for 3 minutes. The extracted cDNA was PCRd and followed the library preparation protocol described in Example 7. The sample was sequenced in Illumina Miseq with 2 million reads. The sequences were split into barcodes containing L1L3 (barcode 1) and L2L4 (barcode 2) and mapped onto the mouse and human genomes. The relative fraction of reads mapping to human and mouse genes are shown in FIG. 17A, showing that ~95% are attributed to the intended species. FIG. 17B shows the sequencing data when the divider was never removed and hence the species were never mixed. Even in this ideal case, there were ~5% genes attributed to the other species.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising:
providing a first nucleic acid comprising a photocleavable linker and a spacer sequence;
applying light to cleave the photocleavable linker to expose a 5′-phosphate and remove the spacer sequence from the nucleic acid; and
attaching a second nucleic acid to the 5′-phosphate.

2. The method of claim 1, wherein the light comprises ultraviolet light.

3. The method of any one of claim 1 or 2, wherein attaching the second nucleic acid comprises ligating the second nucleic acid to the first nucleic acid.

4. The method of any one of claims 1-3, comprising attaching the second nucleic acid to the first nucleic acid sequence using a DNA ligase.

5. The method of any one of claims 1-4, wherein the spacer sequences comprises a oligonucleotide.

6. The method of any one of claims 1-5, wherein the photocleavable linker comprises a structure:

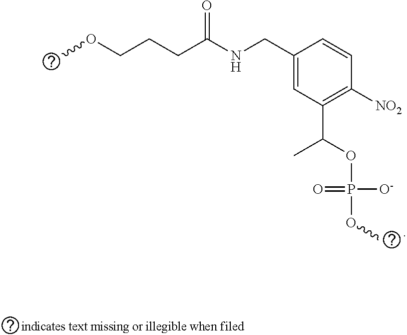

7. The method of any one of claims 1-6, wherein the photocleavable linker comprises a structure formed using:

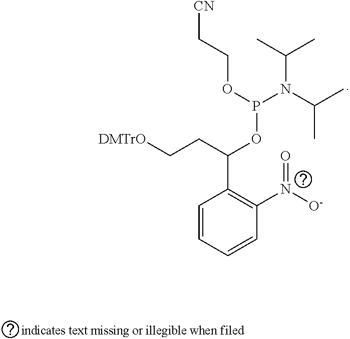

8. The method of any one of claims 1-7, wherein the photocleavable linker comprises a structure formed using:

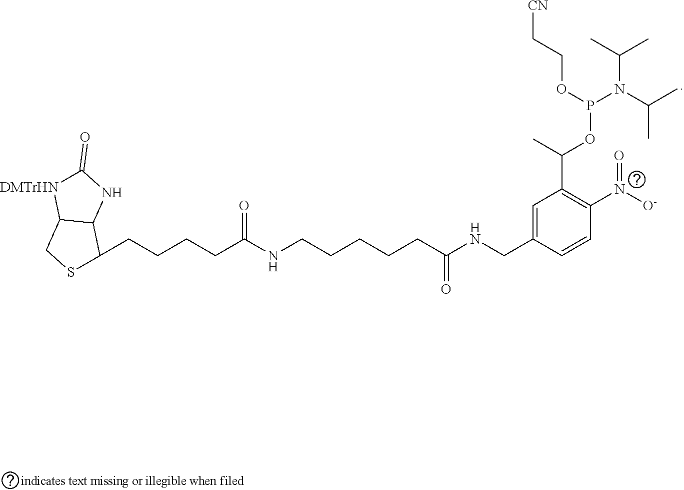

9. The method of any one of claims 1-8, wherein the photocleavable linker comprises a structure formed using:

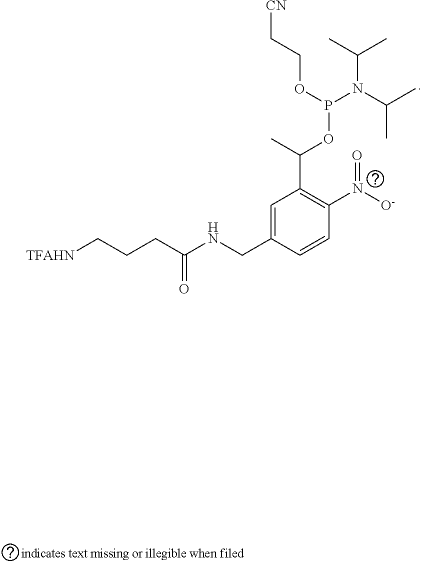

10. The method of any one of claims 1-9, wherein the photocleavable linker comprises a structure formed using:

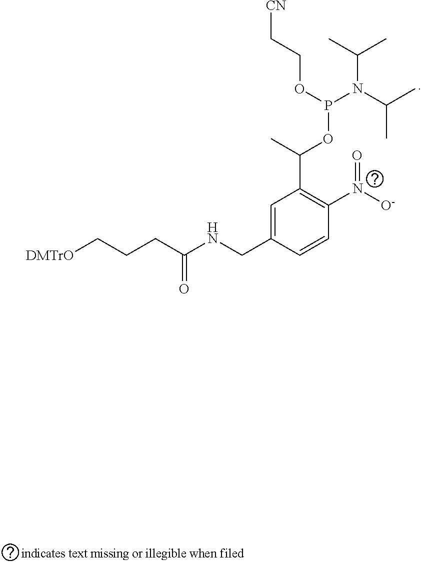

11. The method of any one of claims 1-10, further comprising using a splint DNA to attach the first nucleic acid to the second nucleic acid.

12. The method of any one of claims 1-11, further comprising using a plurality of splint DNA to attach the first nucleic acid to the second nucleic acid.

13. The method of any one of claims 1-12, wherein the second nucleic acid comprises a second photocleavable linker.

14. The method of claim 13, further comprising applying light to cleave the second photocleavable linker to expose a second 5′-phosphate.

15. The method of claim 14, further comprising attaching a third nucleic acid to the second 5′-phosphate.

16. The method of any one of claims 1-15, wherein the first nucleic acid is covalently attached to a sample.

17. The method of any one of claims 1-16, wherein the first nucleic acid is non-covalently attached to a sample.

18. The method of any one of claims 1-17, further comprising sequencing the first nucleic acid and the second nucleic acid.

19. The method of any one of claims 1-18, wherein the first nucleic acid consists of 1 nucleotide.

20. The method of any one of claims 1-19, wherein the first nucleic acid comprises a plurality of nucleotides.

21. The method of any one of claims 1-20, wherein the second nucleic acid consists of 1 nucleotide.

22. The method of any one of claims 1-21, wherein the second nucleic acid comprises a plurality of nucleotides.

23. A method, comprising:
providing a first nucleic acid comprising a photocleavable protecting group;
applying light to cleave the photocleavable protecting group to expose a 5′-OH;
phosphorylating the 5′-OH to a 5′ phosphate; and
attaching a second nucleic acid to the 5′-phosphate.

24. The method of claim 23, wherein the light comprises ultraviolet light.

25. The method of any one of claim 23 or 24, wherein attaching the second nucleic acid comprises ligating the second nucleic acid to the first nucleic acid.

26. The method of any one of claims 23-25, comprising attaching the second nucleic acid to the first nucleic acid sequence using a DNA ligase.

27. The method of any one of claims 23-26, wherein the photocleavable protection group has a structure:

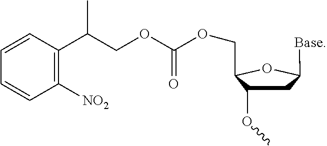

28. The method of any one of claims 23-27, wherein the photocleavable protection group has a structure:

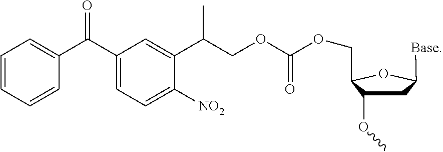

29. The method of any one of claims 23-28, further comprising using a splint DNA to attach the first nucleic acid to the second nucleic acid.

30. The method of any one of claims 23-29, further comprising using a plurality of splint DNA to attach the first nucleic acid to the second nucleic acid.

31. The method of any one of claims 23-30, wherein the second nucleic acid comprises a second photocleavable protection group.

32. The method of claim 31, further comprising applying light to cleave the second photocleavable protection group to expose a second 5′-OH.

33. The method of claim 32, further comprising attaching a third nucleic acid to the second 5′-phosphate.

34. The method of any one of claims 23-33, wherein the first nucleic acid is covalently attached to a sample.

35. The method of any one of claims 23-34, wherein the first nucleic acid is non-covalently attached to a sample.

36. The method of any one of claims 23-35, further comprising sequencing the first nucleic acid and the second nucleic acids.

37. The method of any one of claims 23-36, wherein the first nucleic acid consists of 1 nucleotide.

38. The method of any one of claims 23-37, wherein the first nucleic acid comprises a plurality of nucleotides.

39. The method of any one of claims 23-38, wherein the second nucleic acid consists of 1 nucleotide.

40. The method of any one of claims 23-39, wherein the second nucleic acid comprises a plurality of nucleotides.

41. A method, comprising:
providing a first nucleic acid comprising a first photocleavable blocking group;
applying light to the sample to cleave the first photocleavable blocking group;
attaching a second nucleic acid to the first nucleic acid, wherein the second addition sequence comprises a second photocleavable blocking group;
applying light to the sample to cleave the second photocleavable blocking group; and
attaching a third nucleic acid to the second nucleic acid.

42. A method, comprising:
providing a first nucleic acid comprising a first photocleavable blocking group;
applying light to the sample to cleave the first photocleavable blocking group;
exposing the first nucleic acid to a DNA splint comprising a first portion and a second portion, wherein the first portion binds to at least a portion of the first nucleic acid;
exposing the splint sequence to a second nucleic acid, wherein the second portion binds to at least a portion of the second nucleic acid; and
attaching the first nucleic acid to the second nucleic acid.

43. The method of claim 42, comprising ligating the first nucleic acid to the second nucleic acid.

44. The method of any one of claim 42 or 43, comprising attaching the first nucleic acid to the second nucleic acid using a DNA ligase.

45. The method of claim 44, wherein the first nucleic acid further comprises a spacer sequence that inhibits the DNA ligase from attaching nucleic acids.

46. The method of claim 45, wherein the spacer sequence comprises an oligonucleotide.

47. The method of any one of claims 42-46, wherein exposing the initial sequence to a splint sequence comprises exposing the initial sequence to a plurality of splint sequences each comprising a first portion and a second portion, the plurality of splint sequences including a plurality of different first portions, wherein at least one of the first portions binds to at least a portion of the initial sequence.

48. A method, comprising:
providing a first nucleic acid comprising a photocleavable linker and a spacer sequence;
applying light to cleave the photocleavable linker and remove the spacer sequence from the nucleic acid; and
attaching a second nucleic acid to the first nucleic acid using a DNA ligase, wherein the spacer sequence, when present, inhibits the DNA ligase from attaching nucleic acids.

49. A method, comprising:
attaching a nucleic acid in a first location but not a second location within a sample, wherein the nucleic acid comprises a photocleavable blocking group.

50. A method, comprising:
attaching nucleic acids to different locations within a sample using a DNA ligase, wherein the nucleic acids comprise a blocking group that inhibits the DNA ligase from binding additional nucleotides to the DNA.


SEQUENCE LISTING (73 sequences)

SEQ ID NO: 1 - DNA, 25 bp, synthetic construct
  source 1..25: mol_type = other DNA
tgtgttaggtatggaagagattttt

SEQ ID NO: 2 - DNA, 80 bp, synthetic construct
  source 1..80: mol_type = other DNA
tgtgttaggtatggaagagattttttttttttttttttttttttttttttttttttttttaatcagccataccacatttg

SEQ ID NO: 3 - DNA, 80 bp, synthetic construct
  source 1..80: mol_type = other DNA
agagaatggataggttgtgtttttttttttttttttttttttttttttttttttttttttaatcagccataccacatttg

SEQ ID NO: 4 - DNA, 65 bp, synthetic construct
  source 1..65: mol_type = other DNA
tgtgttaggtatggaagagatttttttttttttttttttttttttaatcagccataccacatttg

SEQ ID NO: 5 - DNA, 20 bp, synthetic construct
  source 1..20: mol_type = other DNA
tttttttttttttttttttt

SEQ ID NO: 6 - DNA, 16 bp, synthetic construct
  source 1..16: mol_type = other DNA
gtaaaacgacggccag

SEQ ID NO: 7 - DNA, 20 bp, synthetic construct
  source 1..20: mol_type = other DNA
ggattattcataccgtccca

SEQ ID NO: 8 - DNA, 20 bp, synthetic construct
  source 1..20: mol_type = other DNA
caaatgtggtatggctgatt

SEQ ID NO: 9 - DNA, 15 bp, synthetic construct
  source 1..15: mol_type = other DNA
acctatccattctct

SEQ ID NO: 10 - DNA, 15 bp, synthetic construct
  source 1..15: mol_type = other DNA
acacatccattctct

SEQ ID NO: 11 - DNA, 15 bp, synthetic construct
  source 1..15: mol_type = other DNA
tccatacctatctct

SEQ ID NO: 12 - DNA, 15 bp, synthetic construct
  source 1..15: mol_type = other DNA
acacaacctatctct

SEQ ID NO: 13 - DNA, 15 bp, synthetic construct
  source 1..15: mol_type = other DNA
tccatacacatctct

SEQ ID NO: 14 - DNA, 15 bp, synthetic construct
  source 1..15: mol_type = other DNA
acctaacacatctct

SEQ ID NO: 15 - DNA, 15 bp, synthetic construct
  source 1..15: mol_type = other DNA
acctatctcttccat

SEQ ID NO: 16 - DNA, 15 bp, synthetic construct
  source 1..15: mol_type = other DNA
acacatctcttccat

SEQ ID NO: 17 - DNA, 15 bp, synthetic construct
  source 1..15: mol_type = other DNA
tctctacctatccat

SEQ ID NO: 18 - DNA, 15 bp, synthetic construct
  source 1..15: mol_type = other DNA
acacaacctatccat

SEQ ID NO: 19 - DNA, 15 bp, synthetic construct
  source 1..15: mol_type = other DNA
tctctacacatccat

SEQ ID NO: 20 - DNA, 15 bp, synthetic construct
  source 1..15: mol_type = other DNA
acctaacacatccat

SEQ ID NO: 21 - DNA, 15 bp, synthetic construct
  source 1..15: mol_type = other DNA
tccattctctaccta

SEQ ID NO: 22 - DNA, 15 bp, synthetic construct
  source 1..15: mol_type = other DNA
acacatctctaccta

SEQ ID NO: 23 - DNA, 15 bp, synthetic construct
  source 1..15: mol_type = other DNA
tctcttccataccta

SEQ ID NO: 24 - DNA, 15 bp, synthetic construct
  source 1..15: mol_type = other DNA
acacatccataccta

SEQ ID NO: 25 - DNA, 15 bp, synthetic construct
  source 1..15: mol_type = other DNA
tctctacacaaccta

SEQ ID NO: 26 - DNA, 15 bp, synthetic construct
  source 1..15: mol_type = other DNA
tccatacacaaccta

SEQ ID NO: 27 - DNA, 15 bp, synthetic construct
  source 1..15: mol_type = other DNA
tccattctctacaca

SEQ ID NO: 28 - DNA, 15 bp, synthetic construct
  source 1..15: mol_type = other DNA
acctatctctacaca

SEQ ID NO: 29 - DNA, 15 bp, synthetic construct
  source 1..15: mol_type = other DNA
tctcttccatacaca

SEQ ID NO: 30 - DNA, 15 bp, synthetic construct
  source 1..15: mol_type = other DNA
acctatccatacaca

SEQ ID NO: 31 - DNA, 15 bp, synthetic construct
  source 1..15: mol_type = other DNA
tctctacctaacaca

SEQ ID NO: 32 - DNA, 15 bp, synthetic construct
  source 1..15: mol_type = other DNA
tccatacctaacaca

SEQ ID NO: 33 - DNA, 20 bp, synthetic construct
  source 1..20: mol_type = other DNA
tccattctcttgggacggta

SEQ ID NO: 34 - DNA, 20 bp, synthetic construct
  source 1..20: mol_type = other DNA
acctatctcttgggacggta

SEQ ID NO: 35 - DNA, 20 bp, synthetic construct
  source 1..20: mol_type = other DNA
acacatctcttgggacggta

SEQ ID NO: 36 - DNA, 20 bp, synthetic construct
  source 1..20: mol_type = other DNA
tctcttccattgggacggta

SEQ ID NO: 37 - DNA, 20 bp, synthetic construct
  source 1..20: mol_type = other DNA
acctatccattgggacggta

SEQ ID NO: 38 - DNA, 20 bp, synthetic construct
  source 1..20: mol_type = other DNA
acacatccattgggacggta

SEQ ID NO: 39 - DNA, 20 bp, synthetic construct
  source 1..20: mol_type = other DNA
tctctacctatgggacggta

SEQ ID NO: 40 - DNA, 20 bp, synthetic construct
  source 1..20: mol_type = other DNA
tccatacctatgggacggta

SEQ ID NO: 41 - DNA, 20 bp, synthetic construct
  source 1..20: mol_type = other DNA
acacaacctatgggacggta

SEQ ID NO: 42 - DNA, 20 bp, synthetic construct
  source 1..20: mol_type = other DNA
tctctacacatgggacggta

SEQ ID NO: 43 - DNA, 20 bp, synthetic construct
  source 1..20: mol_type = other DNA
tccatacacatgggacggta

SEQ ID NO: 44 - DNA, 20 bp, synthetic construct
  source 1..20: mol_type = other DNA
acctaacacatgggacggta

SEQ ID NO: 45 - DNA, 10 bp, synthetic construct
  source 1..10: mol_type = other DNA
agagaagaga

SEQ ID NO: 46 - DNA, 10 bp, synthetic construct
  source 1..10: mol_type = other DNA
aaggtttgga

SEQ ID NO: 47 - DNA, 15 bp, synthetic construct
  source 1..15: mol_type = other DNA
agagatgtgtagaga

SEQ ID NO: 48 - DNA, 15 bp, synthetic construct
  source 1..15: mol_type = other DNA
tgtgttgtgtttgga

SEQ ID NO: 49 - DNA, 10 bp, synthetic construct
  source 1..10: mol_type = other DNA
agagaatgga

SEQ ID NO: 50 - DNA, 10 bp, synthetic construct
  source 1..10: mol_type = other DNA
taggtttgga

SEQ ID NO: 51 - DNA, 15 bp, synthetic construct
  source 1..15: mol_type = other DNA
agagatgtgtatgga

SEQ ID NO: 52 - DNA, 15 bp, synthetic construct
  source 1..15: mol_type = other DNA
tgtgtatggattgga

SEQ ID NO: 53 - DNA, 20 bp, synthetic construct
  source 1..20: mol_type = other DNA
agagatgtgtatggaagaga

SEQ ID NO: 54 - DNA, 25 bp, synthetic construct
  source 1..25: mol_type = other DNA
agagatgtgttgtgttttttttttt

SEQ ID NO: 55 - DNA, 25 bp, synthetic construct
  source 1..25: mol_type = other DNA
ttttttttttttttttttttttttt

SEQ ID NO: 56 - DNA, 15 bp, synthetic construct
  source 1..15: mol_type = other DNA
acacatctcttctct

SEQ ID NO: 57 - DNA, 25 bp, synthetic construct
  source 1..25: mol_type = other DNA
acacaacacatctcttctcttctct

SEQ ID NO: 58 - DNA, 30 bp, synthetic construct
  source 1..30: mol_type = other DNA
tgtgttaggtatggaagagatttttttttt

SEQ ID NO: 59 - DNA, 65 bp, synthetic construct
  source 1..65: mol_type = other DNA
tgtgttaggtatggaagagattttttttttttttttttttttttttttttttttttttttttttt

SEQ ID NO: 60 - DNA, 65 bp, synthetic construct
  source 1..65: mol_type = other DNA
agagaatggataggttgtgtaatcagccataccacatttgttttttttttttttttttttttttt

SEQ ID NO: 61 - DNA, 20 bp, synthetic construct
  source 1..20: mol_type = other DNA
agagaatggataggttgtgt

SEQ ID NO: 62 - DNA, 20 bp, synthetic construct
  source 1..20: mol_type = other DNA
atggaagagatgtgttaggt

SEQ ID NO: 63 - DNA, 20 bp, synthetic construct
  source 1..20: mol_type = other DNA
taggttgtgtagagaatgga

SEQ ID NO: 64 - DNA, 20 bp, synthetic construct
  source 1..20: mol_type = other DNA
tgtgttaggtatggaagaga

SEQ ID NO: 65 - DNA, 15 bp, synthetic construct
  source 1..15: mol_type = other DNA
tccattctcttccat

SEQ ID NO: 66 - DNA, 20 bp, synthetic construct
  source 1..20: mol_type = other DNA
aatcagccataccacatttg

SEQ ID NO: 67 - DNA, 20 bp, synthetic construct
  source 1..20: mol_type = other DNA
gatccgattggaaccgtccc

SEQ ID NO: 68 - DNA, 71 bp, synthetic construct
  source 1..71: mol_type = other DNA
acgagcatcagcagcatacgannnnnnnnnnnnnnnnnnnntttttttttttttttttttttttttttttt

SEQ ID NO: 69 - DNA, 72 bp, synthetic construct
  source 1..72: mol_type = other DNA
agagaatggataggttgtgtnnnnnnnnnnnnnnnnnnnnttttttttttttttttttttttttttttttvn

SEQ ID NO: 70 - DNA, 30 bp, synthetic construct
  source 1..30: mol_type = other DNA
  misc_feature 28..29: note = RNA
  misc_feature 1..27: note = DNA
  misc_feature 30: note = DNA
aagcagtggtatcaacgcagagtacatggg

SEQ ID NO: 71 - DNA, 34 bp, synthetic construct
  source 1..34: mol_type = other DNA
gtctcgtgggctcggagatgtgtataagagacag

SEQ ID NO: 72 - DNA, 54 bp, synthetic construct
  source 1..54: mol_type = other DNA
tcgtcggcagcgtcagatgtgtataagagacagacgagcatcagcagcatacga

SEQ ID NO: 73 - DNA, 21 bp, synthetic construct
  source 1..21: mol_type = other DNA
acgagcatcagcagcatacga